(19) United States
(12) Patent Application Publication
(10) Pub. No.: US 2025/0204799 A1
(43) Pub. Date: Jun. 26, 2025

(54) MEASURING DEVICE AND MEASURING METHOD

(71) Applicant: Sharp Kabushiki Kaisha, Osaka (JP)

(72) Inventors: RYOTA TOMIZAWA, Osaka (JP); Yoshihisa Adachi, Osaka (JP); Azusa Nakano, Osaka (JP)

(21) Appl. No.: 18/976,420

(22) Filed: Dec. 11, 2024

(30) Foreign Application Priority Data
Dec. 21, 2023 (JP) 2023-215333

(51) Int. Cl. A61B 5/0255; A61B 5/00; G06T 7/00; G06T 7/90

(52) U.S. Cl. CPC A61B 5/0255; A61B 5/0077; G06T 7/0016; G06T 7/90; G06T 2207/10016; G06T 2207/30201

(57) ABSTRACT

A measuring device includes: a capturing unit that captures a biological subject to sequentially obtain a plurality of images; and a time point estimating unit that estimates a time point at which each of the plurality of images is obtained in accordance with a periodic variation that appears in a sequence of a plurality of pixel values each obtained from a corresponding one of the plurality of images.

### CROSS-REFERENCE TO RELATED APPLICATION

The present application claims priority from Japanese Application JP2023-215333, filed on Dec. 21, 2023, the content of which is hereby incorporated by reference into this application.

### BACKGROUND

### 1. Field

The present disclosure relates to a measuring device and a measuring method.

### 2. Description of the Related Art

Japanese Unexamined Patent Application Publication No. 2021-048527 discloses a technique to output a video image including a frame image dated with a character string indicating the date and time when the frame image was captured.

### SUMMARY

In the technique disclosed in the Japanese Unexamined Patent Application Publication No. 2021-048527, a camera generates a frame image dated with a character string indicating the date and time when the frame image was captured, and presents the user the date and time when each of the frame images was captured. Hence, when the technique disclosed in the Japanese Unexamined Patent Application Publication No. 2021-048527 is used, some of the frame images in an angle of view of the camera are missing because of the character string indicating the date and time of the capturing. Hence, an aspect of the present disclosure sets out to provide a measuring device and a measuring method capable of identifying the date and time when the images are obtained with none of the obtained images missing.

A measuring device according to an aspect of the present disclosure includes: a capturing unit that captures a biological subject to sequentially obtain a plurality of images; and a time point estimating unit that estimates a time point at which each of the plurality of images is obtained in accordance with a periodic variation that appears in a sequence of a plurality of pixel values each obtained from a corresponding one of the plurality of images.

A measuring method according to an aspect of the present disclosure includes: a step of capturing a biological subject to sequentially obtain a plurality of images; and a step of estimating a time point at which each of the plurality of images is obtained in accordance with a periodic variation that appears in a sequence of a plurality of pixel values each obtained from a corresponding one of the plurality of images.

### BRIEF DESCRIPTION OF THE DRAWINGS

FIG. 1 is a view showing an example of how to use a measuring device;

FIG. 2 is a block diagram illustrating an exemplary configuration of the measuring device according to a first embodiment;

FIG. 3 is a flowchart showing an exemplary operation performed on the measuring device according to the first embodiment;

FIG. 4 is a flowchart showing the exemplary operation succeeding FIG. 3 and performed on the measuring device according to the first embodiment;

FIG. 5 is a diagram illustrating an exemplary case where a frame drop has occurred because processing time periods vary;

FIG. 6 is a diagram illustrating an example of a case where a frame drop occurs because the processing time period is longer than an exposure time;

FIG. 7 is a graph showing an example of time-series variations of a representative value of pixel values of pixels in a first region of interest;

FIG. 8 is a graph showing an example of: the time-series variations of the representative value among the pixel values of the pixels in the first region of interest; and an assumed variation signal;

FIG. 9 is a graph showing an exemplary case where a frame number of a capture image is assigned to an image for which the representative value exemplified in FIG. 8 is calculated;

FIG. 10 is a block diagram illustrating an exemplary configuration of the measuring device according to a second embodiment;

FIG. 11 is a flowchart showing an exemplary operation performed on the measuring device according to the second embodiment; and

FIG. 12 is a flowchart showing the exemplary operation succeeding FIG. 11 and performed on the measuring device according to the second embodiment.

### DETAILED DESCRIPTION OF THE DISCLOSURE

### Fifth Embodiment

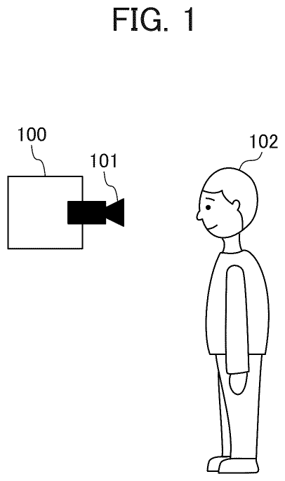

A first embodiment will be described below with reference to FIGS. 1 to 9. Note that, throughout the drawings, like reference signs denote identical or similar constituent features. Such features will not be repeatedly elaborated upon.

FIG. 1 is a view showing an example of how to use a measuring device 100. As exemplified in FIG. 1, the measuring device 100 includes a capturing unit 101.

The measuring device 100 causes the capturing unit 101 to capture a biological subject 102, and calculates a biological signal from a plurality of images obtained by the capturing unit 101. For example, the measuring device 100 is a personal computer (PC), a smartphone, a tablet terminal, and a pulse wave estimating terminal. For example, the biological signal is a pulse wave signal. In this specification, the pulse wave is a time-series signal indicating variations in the volume of a blood vessel. The pulse wave is calculated, for a single position on a body surface, from a time-series signal indicating a pixel value of a pixel included in an image. In this specification, the pixel value is information indicating brightness of a pixel included in an image. For example, the pixel value is either a pixel value, or a luminance value, for each of the pixels in red (R), green (G), and blue (B).

For example, the capturing unit 101 is either a charge coupled device (CCD), or a complementary metal oxide semiconductor (CMOS) image sensor. The capturing unit 101 may also be an image sensor for a camera including a red-green-blue (RGB) filter.

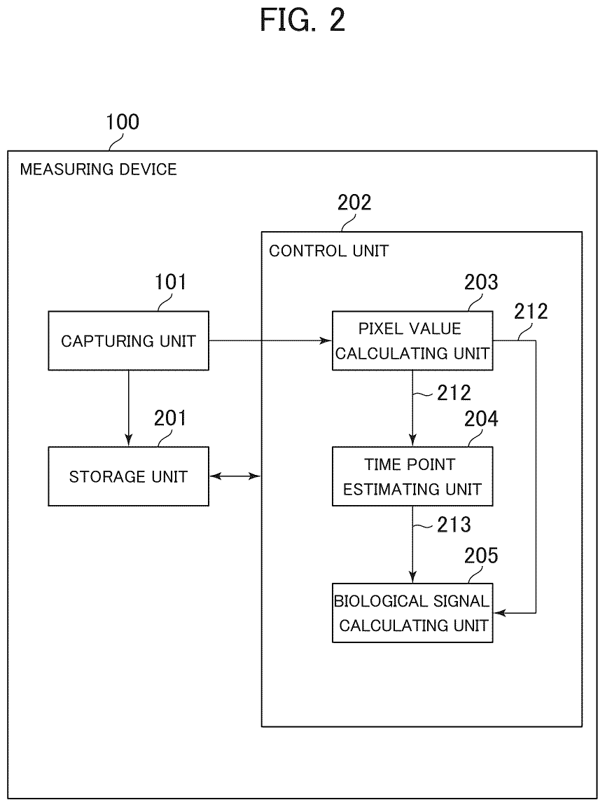

FIG. 2 is a block diagram illustrating an exemplary configuration of the measuring device 100 according to this embodiment. The measuring device 100 includes: the capturing unit 101; a storage unit 201; and a control unit 202.

The capturing unit 101 captures the biological subject 102 to sequentially obtain a plurality of images. Specifically, the capturing unit 101 captures the biological subject 102 to sequentially obtain a plurality of capture images at a frame rate Fr. The frame rate Fr is set to cause aliasing due to a blink that a lighting fixture makes regularly. Note that the capturing unit 101 may obtain the plurality of capture images at regular time intervals in different lengths. The obtained capture images include an image of the body surface of the biological subject 102. Examples of the image of the body surface include an image of a face, an image of a cheek, an image of a forehead, an image of a palm, an image of a wrist, and an image of a sole. For example, the regular blink of the lighting fixture is caused when the lighting fixture flickers.

The storage unit 201 is a recording medium capable of recording, for example, various data and programs. The storage unit 201 is, for example, a hard disk, a solid state drive (SSD), and a semiconductor memory.

The control unit 202 executes various processes in accordance with the programs and the data stored in the storage unit 201. The control unit 202 is implemented in the form of, for example, a processor such as a central processing unit (CPU). The control unit 202 includes: a pixel value calculating unit 203; a time point estimating unit 204; and a biological signal calculating unit 205.

The pixel value calculating unit 203 calculates a first representative pixel value 212 among pixel values of two or more pixels in a first region of interest, from each of a plurality of processed images IMG that are at least partially the plurality of capture images obtained by the capturing unit 101. Specifically, the pixel value calculating unit 203 sequentially obtains latest capture images as the processed images IMG from among the plurality of capture images obtained by the capturing unit 101. Then, the pixel value calculating unit 203 calculates, from each of the processed images IMG, the first representative pixel value 212 among the pixel values of two or more pixels in the first region of interest. Hence, the pixel value calculating unit 203 calculates each of a plurality of the first representative pixel values 212 from a corresponding one of the plurality of the processed images IMG. The first region of interest according to this embodiment includes an image of the body surface of the biological subject 102. The pixel value calculating unit 203 calculates the first representative pixel value 212 from the processed image IMG. After that, the pixel value calculating unit 203 obtains a succeeding processed image IMG from the capturing unit 101.

The time point estimating unit 204 estimates a time point 213 at which each of the plurality of processed images IMG is obtained by the capturing unit 101, in accordance with a periodic variation that appears in a sequence of the plurality of pixel values each obtained from a corresponding one of the plurality of processed images IMG. Specifically, the sequence of the plurality of pixel values is a sequence of the plurality of first representative pixel values 212.

The biological signal calculating unit 205 calculates a biological signal from the plurality of first representative pixel values 212.

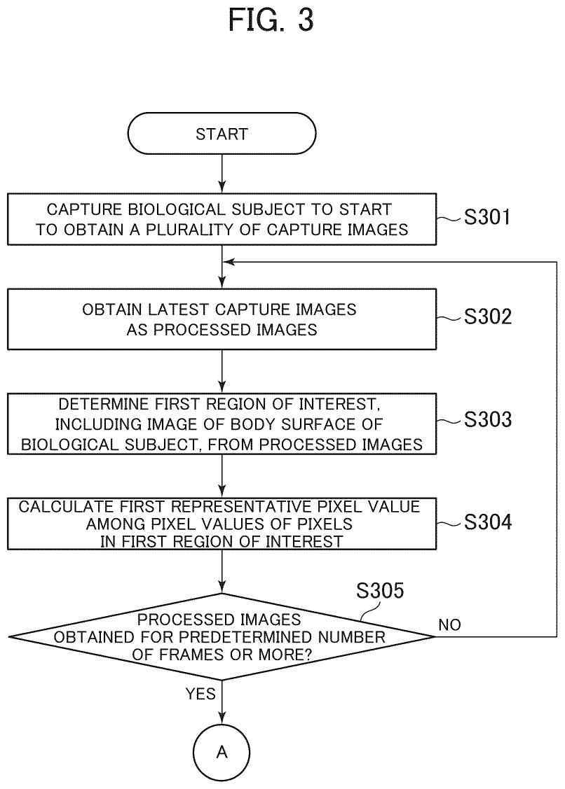

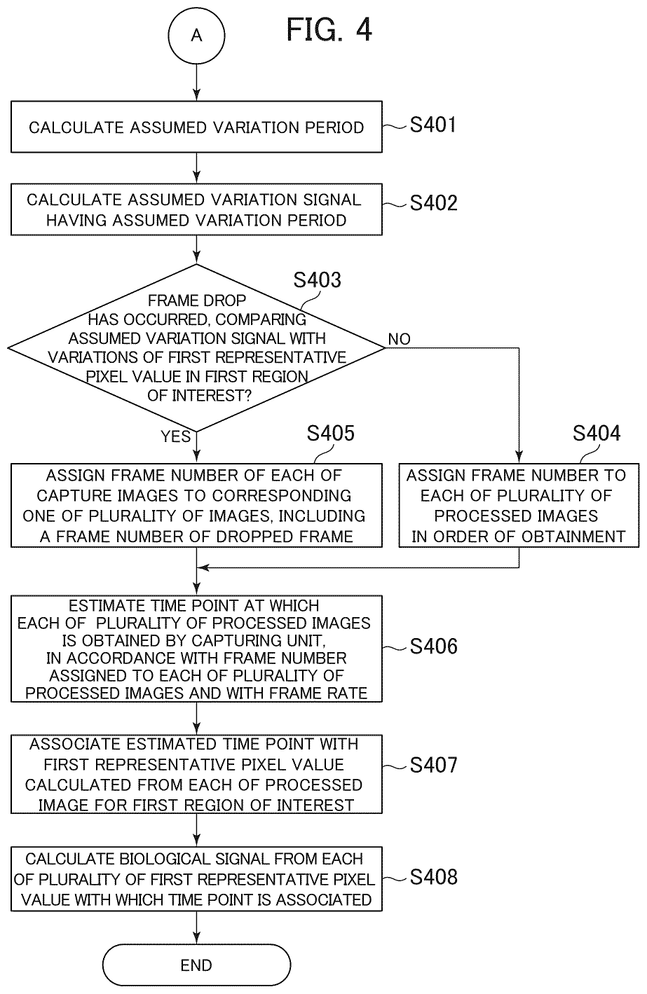

FIGS. 3 and 4 are flowcharts showing an exemplary operation performed on the measuring device 100 according to this embodiment.

At Step S301, the capturing unit 101 captures the biological subject 102 at the frame rate Fr to start to obtain a moving image. That is, the capturing unit 101 captures the biological subject 102 at the frame rate Fr to start to sequentially obtain a plurality of capture images in time series. The obtained capture images include an image of the body surface of the biological subject 102.

At Step S302, the pixel value calculating unit 203 obtains latest capture images as the processed images IMG from among the plurality of capture images obtained by the capturing unit 101. Specifically, the pixel value calculating unit 203 requests the obtained latest capture images from the capturing unit 101. In response to the request from the pixel value calculating unit 203, the capturing unit 101 transmits the latest capture images to the pixel value calculating unit 203. The pixel value calculating unit 203 obtains, as the processed images IMG, the capture images transmitted from the capturing unit 101. Then, the pixel value calculating unit 203 causes the storage unit 210 to sequentially store the obtained processed images IMG. Note that, if the measuring device 100 includes an internal clock (not shown) that measures a time point indicated by hours, minutes, and seconds, the pixel value calculating unit 203 may associate, with a processed image IMG for a first frame, a time point measured with the internal clock when the processed image IMG for the first frame is obtained, and cause the storage unit 201 to store the processed image IMG for the first frame.

At Step S303, the pixel value calculating unit 203 determines the first region of interest from the processed images IMG obtained at Step S302. The first region of interest includes an image of the body surface and a plurality of pixels. For example, if the processed images IMG include an image of a face of the biological subject 102, the first region of interest includes an image of the cheek, an image of the forehead, or an image of the glabella. The first region of interest may include either one first region of interest, or a plurality of first regions of interest. The first region of interest may have either a polygonal shape surrounded with straight lines, or a shape surrounded with a curved line. Alternatively, the first region of interest may be a closed region formed of a straight line and a curved line.

At Step S304, the pixel value calculating unit 203 calculates the first representative pixel value 212 among pixel values of pixels in the first region of interest determined at Step S303. For example, the first representative pixel value 212 is an average value, a median value, or a mode value of the pixel values of the pixels in the first region of interest. For example, if the capturing unit 101 is an image sensor for a camera including a red-green-blue (RGB) filter, the pixel value calculating unit 203 may calculate the first representative pixel value 212 among the pixel values of the pixels for each of R, G, and B in the first region of interest.

At Step S305, the pixel value calculating unit 203 determines whether the processed images IMG are obtained for a predetermined number of frames or more. The predetermined number of frames are a number of frames that are necessary and sufficient for calculating a biological signal at Step S408 to be shown later as an example in FIG. 4.

At Step S305, if the processed images IMG are not obtained for the predetermined number of frames or more, the control unit 202 returns the process back to Step S302, and continues the process. That is, if, at Step S305, the processed images IMG are not obtained for the predetermined number of frames or more, the pixel value calculating unit 203 obtains as new processed images IMG the latest capture images obtained at Step S302 by the capturing unit 101. Note that, while the control unit 202 executes the processes of Steps S302 to S305, the capturing unit 101 continues the process of capturing the biological subject 102 at the frame rate Fr and sequentially obtaining a plurality of capture images. Whereas, at Step S305, if the processed images IMG are obtained for the predetermined number of frames or more, the control unit 202 proceeds with the process to Step S401 exemplified in FIG. 4.

Subsequently described below with reference to FIG. 4 will be the operation of the measuring device 100 according to this embodiment.

In an environment in which a lighting fixture regularly blinks, when the capturing unit 101 captures the biological subject 102 to obtain a capture image, a pixel value of a pixel included in the obtained capture image periodically varies. In an environment in which a lighting fixture regularly blinks, when a periodic signal is observed at a frequency different from that of the signal, a signal referred to as aliasing is observed. Hence, when the lighting fixture regularly blinks, and the frame rate Fr is different from a frequency at which the lighting fixture regularly blinks, the aliasing is periodically observed of a signal indicating a time-series pixel value.

Specifically, in an environment in which the lighting fixture regularly blinks at a frequency of Fq[Hz], an aliasing of Fq−n×Fr[Hz] is observed in an image for an n-th frame obtained by the capturing unit 101 at a frame rate of Fr[fps]. When the aliasing periodically occurs, the pixel values of the pixels included in the capture images obtained by the capturing unit 101 are assumed to periodically vary in time series in accordance with the frequency of the aliasing. As a result, if the pixel value calculating unit 203 obtains a processed image IMG when the aliasing occurs, the first representative pixel value 212 is assumed to periodically vary. That is, if the pixel value calculating unit 203 obtains a processed image IMG when the aliasing occurs, the periodical variation includes a frequency component due to the regular blink of the lighting fixture.

Hence, if the capturing unit 101 captures the biological subject 102 and obtains capture images at the frame rate Fr at which the aliasing can be observed, the time point estimating unit 204 can estimate the time point 213 at which an image, namely a processed image IMG, is obtained by the capturing unit 101. The time point 213 is estimated from the period in which the first representative pixel value 212 varies in time series because of the variations of the first representative pixel value 212 by the aliasing. Note that the frame rate Fr is set so that the frequency component of the aliasing is different from a frequency component included in the biological signal.

The frequency component included in the biological signal is a frequency component indicating variations in pixel value in accordance with variations in the volume of the blood vessel of the biological subject 102.

For example, in a region in which an AC power supply is 100 Hz, a lighting fixture causes a flicker of 100 Hz and blinks. When the lighting fixture causes a flicker of 100 Hz, if the frame rate Fr is 50 fps, the aliasing does not occur. Thus, the first representative pixel value 212 does not vary. Hence, the frame rate Fr of 50 fps is not a suitable value for the time point estimating unit 204 to estimate the time point 213 at which a processed image IMG is obtained by the capturing unit 101.

Thus, at Step S401, the time point estimating unit 204 calculates an assumed variation period; that is, a period assumed for the variations of the first representative pixel value 212. For example, in accordance with the frame rate Fr, and with the frequency at which the lighting fixture regularly blinks, the time point estimating unit 204 calculates the assumed variation period; that is, a period in periodic variations, for the pixel values of the plurality of pixels included in each of the plurality of processed images IMG. Alternatively, for example, the time point estimating unit 204 may calculate the assumed variation period to match the time-series variations of the first representative pixel value 212 calculated at Step S304 exemplified in FIG. 3.

At Step S402, the time point estimating unit 204 calculates an assumed variation signal 801 (see FIG. 8); that is, a signal having the assumed variation period calculated at Step S401. For example, the assumed variation signal 801 is a sinusoidal wave having: the assumed variation period calculated; and a phase and an amplitude that match the variations of the first representative pixel value 212 calculated at Step S304 illustrated in FIG. 3.

At Step S403, the time point estimating unit 204 compares the assumed variation signal 801 calculated at Step S402 with the time-series variations of the first representative pixel value 212 to determine whether a frame drop has occurred.

For example, when the pixel value calculating unit 203 executes the processes of Steps S303 and S304, the processing time period is likely to vary depending on how the biological subject 102 looks. Furthermore, for example, if the measuring device 100 is either a personal computer (PC) or a smartphone, the control unit 202 executes a plurality of processes in parallel on a plurality of different application programs. Hence, in the processes at Steps S303 and S304, the processing time periods might vary for each of the processed images IMG.

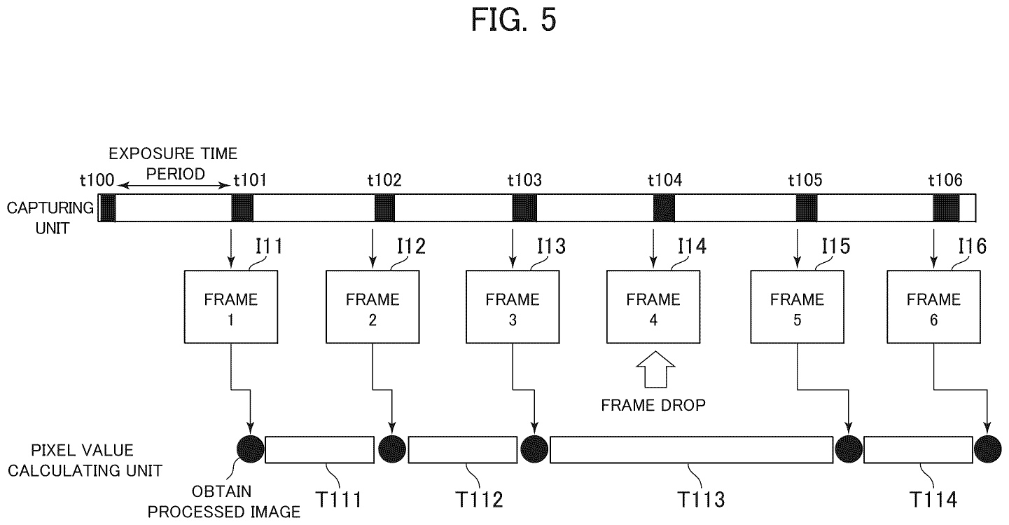

If the processing time periods vary in the processes at Steps S303 and S304, as exemplified in FIG. 5, the pixel value calculating unit 203 cannot obtain any of the capture images obtained by the capturing unit 101 as the processed images IMG, and a frame drop might occur in the processes executed by the pixel value calculating unit 203.

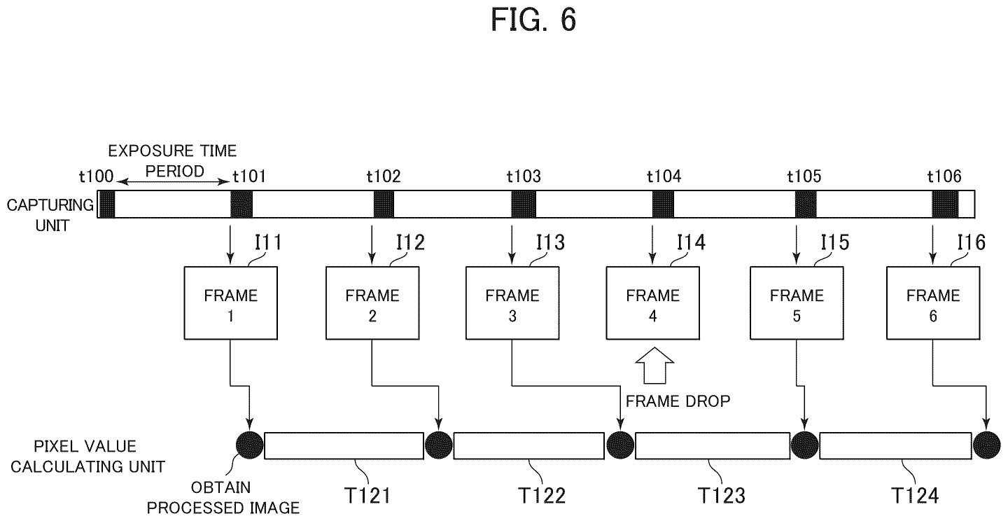

Furthermore, even if the processing time period is constant in the processes of Steps S303 and S304 for each of the processed images IMG, as exemplified in FIG. 6, the processing time period for each of Steps S302 to S305 could be longer than an exposure time period of the capturing unit 101. In such a case, the pixel value calculating unit 203 generates a capture image obtained not as a processed image IMG, and thus a frame drop might occur.

Thus, for example, for the first representative pixel value 212 calculated from each of the processed images IMG, the time point estimating unit 204 determines whether a minimum value of a difference between the assumed variation signal 801 and the first representative pixel value 212 exceeds a threshold value, in the order of the processed images IMG obtained by the pixel value calculating unit 203. If the minimum value of the difference between the assumed variation signal 801 and the first representative pixel value 212 does not exceed the threshold value, the time point estimating unit 204 determines that a frame drop has not occurred to the processed image IMG for which the first representative pixel value 212 is calculated. Whereas, if the minimum value of the difference between the assumed variation signal 801 and the first representative pixel value 212 exceeds the threshold value, the time point estimating unit 204 determines that a frame drop has occurred immediately before the processed image IMG for which the first representative pixel value 212 is calculated.

For example, suppose a case where the frame rate Fr is 55 fps when the lighting fixture produces a flicker of 100 Hz and regularly blinks. If the first representative pixel value 212 varies at a frame period of 5.5; that is, an assumed variation period, the time point estimating unit 204 determines at Step S403 that no frame drop has occurred to the plurality of processed images IMG from the plurality of capture images obtained by the capturing unit 101.

Whereas, suppose a case where the frame rate Fr is 55 fps when the lighting fixture produces a flicker of 100 Hz regularly blinks. If the first representative pixel value 212 does not vary at a frame period of 5.5; that is, an assumed variation period, the time point estimating unit 204 determines at Step S403 that a frame drop has occurred to the plurality of processed images IMG from the plurality of capture images obtained by the capturing unit 101. That is, the time point estimating unit 204 calculates the assumed variation period in accordance with the frame rate Fr and the frequency at which the lighting fixture regularly blinks. Then, if the assumed variation signal having the assumed variation period calculated does not match the plurality of first representative pixel values 212, the time point estimating unit 204 determines that a frame drop has occurred.

At Step S403, if the time point estimating unit 204 determines that a frame drop has not occurred, the time point estimating unit 204 assigns at Step S404 a frame number to each of the plurality of processed images IMG in the order of obtainment. Then, the control unit 202 proceeds with the process to Step S406.

Whereas, at Step S403, if the time point estimating unit 204 determines that a frame drop has occurred, the time point estimating unit 204 respectively assigns at Step S405 frame numbers of the captured images to processed images IMG, the frame numbers including a frame number where a frame drop occurs and the first representative pixel value 212 is not calculated. Then, the control unit 202 proceeds with the process to Step S406.

At Step S406, the time point estimating unit 204 estimates the time point 213 at which each of the plurality of processed images IMG is obtained by the capturing unit 101, in accordance with a frame number assigned to each of the plurality of processed images IMG and with the frame rate Fr.

In accordance with a period of aliasing, the time point estimating unit 204 estimates the time point 213 at which each of the plurality of images is obtained by the capturing unit 101. Hence, when the capturing unit 101 obtains capture images at the frame rate Fr at which the aliasing can be observed, the time point estimating unit 204 can estimate, from a period in which the first representative pixel value 212 varies, the time point 213 at which each processed image IMG is obtained by the capturing unit 101.

For example, if a time point measured by the internal clock is associated with a processed image IMG1 for the first frame, the time point estimating unit 204 uses as a starting point an absolute time point associated with the processed image IMG1 for the first frame, and estimates an absolute time point indicating a time point at which a processed image IMG for the n-th frame is obtained by the capturing unit 101. The absolute time point is estimated by a product of an exposure time period determined by the frame rate Fr and the frame number n. Here, n is a natural number of 2 or more.

Alternatively, the time point estimating unit 204 may use as the starting point a time point at which the processed image IMG for the first frame is obtained, and estimate a relative time point indicating an elapsed time period until a time point at which a processed image IMG for the n-th frame is obtained by the capturing unit 101. The relative time point may be estimated by the product of the exposure time period determined by the frame rate and the frame number n.

At Step S407, the time point estimating unit 204 associates the time point 213 estimated at Step S406 with the first representative pixel value 212 calculated from each of the processed images IMG. Hence, because of the processes at Steps S401 to S407, the measuring device 100 according to this embodiment can identify, for the processed images, the date and time when images are obtained appropriately with none of the obtained images missing.

At Step S408, the biological signal calculating unit 205 calculates a biological signal from each of the plurality of first representative pixel values 212 with which the time point 213 is associated. For example, the biological signal calculating unit 205 processes a signal indicating temporal variations of the first representative pixel value 212 by independent component analysis and pigment component separation, and calculates the processing result as a biological signal. The temporal variations of the first representative pixel value 212 include information on variations in the volume of the blood vessel. For example, the biological signal indicates a pulse wave signal.

Note that if the biological signal calculating unit 205 performs such processing as digital filtering and trend removal on the biological signal in order to remove noise from the calculated biological signal, the biological signal calculating unit 205 preferably associates a time point at an equal time interval with a value indicated by a signal representing temporal variations of the first representative pixel value 212. Hence, the biological signal calculating unit 205 may interpolate a value of a frame drop portion in the biological signal, in accordance with each time point 213 associated with a corresponding one of the plurality of processed images IMG. For example, the biological signal calculating unit 205 interpolates the first representative pixel value 212 of a frame drop portion, and processes the signal in which the first representative pixel value 212 of the frame drop portion is interpolated. Hence, the biological signal calculating unit 205 interpolates the value of the frame drop portion in the biological signal.

Alternatively, the biological signal calculating unit 205 may perform either upsampling or downsampling on a value indicated by a signal representing temporal variations of the first representative pixel value 212 in accordance with each time point 213 associated with a corresponding one of the plurality of the processed images IMG, in order to correct the signal representing the temporal variations of the first representative pixel value 212 so that the signal has an equal time interval. As can be seen, the biological signal calculating unit 205 interpolates a value of a frame drop portion in the biological signal, thereby successfully calculating a biological signal more accurately.

Note that, as to the biological signal output by the biological signal calculating unit 205, the time point 213 estimated by the time point estimating unit 204 may be associated with a value indicated by the biological signal. In such a case, the biological signal output by the biological signal calculating unit 205 may have a frame drop not at an equal time interval.

FIG. 5 is a diagram illustrating an exemplary case where a frame drop has occurred because processing time periods vary in the processes at Steps S303 and S304 exemplified in FIG. 3.

At Step S301 exemplified in FIG. 3, the capturing unit 101 starts at a time point t100 a process to obtain a moving image at the frame rate Fr. At time points t101 to t106, the capturing unit 101 captures the biological subject 102 to sequentially obtain capture images I11 to I16. Intervals between the time points of t100 to t106 are exposure time periods determined by the frame rate Fr.

For example, the pixel value calculating unit 203 obtains the image I11 as a processed image IMG for the first frame. Then, the pixel value calculating unit 203 calculates the first representative pixel value 212 from the processed image IMG for the first frame. After that, the pixel value calculating unit 203 obtains a latest capture image I12 as a processed image IMG for a second frame.

Each of a time period T111 to a time period T114 indicates a processing time period for a corresponding one of the processed images IMG required for the processes at Steps S303 and S304. For example, if the time period T111 is shorter than the exposure time period determined by the frame rate Fr, the pixel value calculating unit 203 calculates the first representative pixel value 212 from the processed image IMG for the first frame; namely, the capture image I11, and the capturing unit 101 obtains the capture image I12. After that, the pixel value calculating unit 203 obtains the latest capture image I12 obtained at the time point t102 as the processed image IMG for the second frame.

Likewise, for example, if the time period T112 is shorter than the exposure time period determined by the frame rate Fr, the pixel value calculating unit 203 calculates the first representative pixel value 212 from the processed image IMG for the second frame; namely, the capture image I12, and the capturing unit 101 obtains the capture image I13. After that, the pixel value calculating unit 203 obtains the latest capture image I13 obtained at the time point t103 as the processed image IMG for the third frame.

Whereas, for example, if the time period T113 is longer than the exposure time period determined by the frame rate Fr, the capturing unit 101 obtains the capture image I14 and the capture image I15 before the pixel value calculating unit 203 calculates the first representative pixel value 212 from a processed image IMG for a third frame; that is, the capture image I13 obtained at the time point 103. Hence, the pixel value calculating unit 203 does not obtain, as a processed image IMG, the capture image I14 obtained at the time point t104, and obtains, as a processed image IMG for a fourth frame, the latest capture image I15 obtained at the time point t105.

Thus, if the processing time periods vary in the processes at Steps S303 and S304, the pixel value calculating unit 203 cannot obtain any of the capture images obtained by the capturing unit 101 as the processed images IMG, and a frame drop might occur in the processes executed by the pixel value calculating unit 203.

FIG. 6 is a diagram illustrating an exemplary case where a frame drop has occurred because the processing time period determined by the frame rate Fr is longer than a processing time period for each of the processes at Steps S303 and S304 exemplified in FIG. 3. The time points t100 to t106 and the capture images I11 to 116 are the same as the time points t100 to t106 and the capture images I11 to 116 illustrated in FIG. 5, and thus detailed description thereof will be omitted. Each of a time period T121 to a time period T124 indicates a processing time period of a process at Steps S303 to S304 for a processed image IMG. The processing time period is longer than the exposure time period determined by the frame rate Fr.

For example, each of the time periods T121 to T123 is longer the exposure time period determined by the frame rate Fr, such that, in the time period T123, the capturing unit 101 is to obtain the capture image I14 and the capture image I15. In such a case, the pixel value calculating unit 203 calculates the first representative pixel value 212 from the processed image IMG for the third frame; namely, the capture image I13 obtained at time point t103. After that, the pixel value calculating unit 203 cannot obtain, as a processed image IMG, the capture image I14 obtained at the time point t104, and obtains, as the processed image IMG for the fourth frame, the latest capture image I15 obtained at the time point t105.

Thus, if each of the processing time periods for Steps S303 and S304 is longer than the exposure time period, the pixel value calculating unit 203 cannot obtain any of the capture images obtained by the capturing unit 101 as the processed images IMG, and a frame drop might occur in the processes executed by the pixel value calculating unit 203.

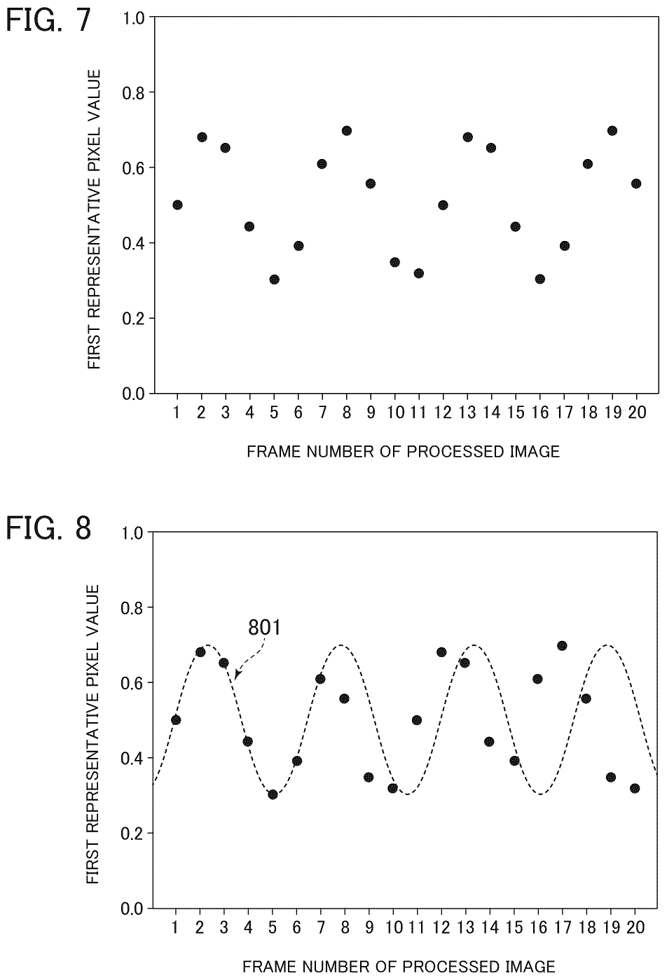

FIG. 7 is a graph showing an example of time-series variations of the first representative pixel value 212. In FIG. 7, the horizontal axis represents the frame numbers assigned to the processed images IMG, and the vertical axis represents the first representative pixel value 212.

For example, if the frame rate Fr is 55 fps when the lighting fixture produces a flicker of 100 Hz and regularly blinks, an aliasing of 10 Hz occurs. In such a case, as illustrated in FIG. 7, the first representative pixel value 212 varies at an assumed variation period of 5.5 frames. If the first representative pixel value 212 varies at the assumed variation period, a frame drop does not occur in the process executed by the pixel value calculating unit 203.

FIG. 8 is a graph showing an example of: time-series variations of the first representative pixel value 212; and an assumed variation signal 801. In FIG. 8, the horizontal axis represents the frame numbers assigned to the processed images IMG, and the vertical axis represents the first representative pixel value 212.

If a frame drop occurs in the process executed by the pixel value calculating unit 203, the assumed variation signal 801 and the plurality of first representative pixel values 212 do not match each other. For example, if the frame rate Fr is 55 fps when the lighting fixture produces a flicker of 100 Hz and regularly blinks, the assumed variation signal 801 is a sinusoidal wave having an assumed variation period of 5.5 frames.

If a frame drop occurs, as exemplified in FIG. 8, the assumed variation signal 801 and the plurality of first representative pixel values 212 do not match each other. Specifically, suppose a case where the pixel value calculating unit 203 does not obtain a capture image for an eighth frame as a processed image IMG, and a frame drop occurs to the capture image for the eighth frame. As illustrated in FIG. 8, a difference is produced between the assumed variation signal 801 and the first representative pixel value 212 calculated from the processed image IMG for the eighth frame.

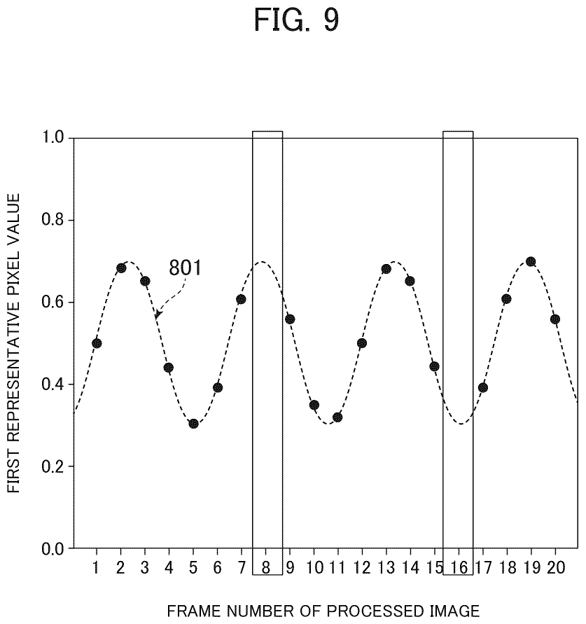

FIG. 9 is a graph showing an exemplary case where a frame number of a capture image is assigned to a processed image IMG for which the first representative pixel value 212 exemplified in FIG. 8 is calculated.

For example, if a frame drop occurs to the capture image for the eighth frame and a capture image for a sixteenth frame, as illustrated in FIG. 9, frame numbers for the eighth frame and the sixteenth frame are added as frame numbers with which the first representative pixel value 212 is not calculated. Thus, frame numbers are assigned to the plurality of respective processed images

IMG. Then, at Step S407 exemplified in FIG. 4, the time point estimating unit 204 estimates a time point at which each of the plurality of processed images IMG is obtained by the capturing unit 101, in accordance with the frame numbers assigned to the plurality of respective processed images IMG and with the frame rate Fr. Then, in accordance with the time point estimated by the time point estimating unit 204, the biological signal calculating unit 205 interpolates values: included in the values indicated by a biological signal; and representing the eighth frame and the sixteenth frame to which the frame drop has occurred. Thanks to such a feature, the biological signal calculating unit 205 can calculate the biological signal more accurately.

As can be seen, the measuring device 100 according to this embodiment can identify the date and time when the images are obtained appropriately with none of obtained images missing.

Hence, even if a frame drop occurs, the measuring device 100 can reduce an effect of the frame drop and calculate a biological signal.

### Second Embodiment

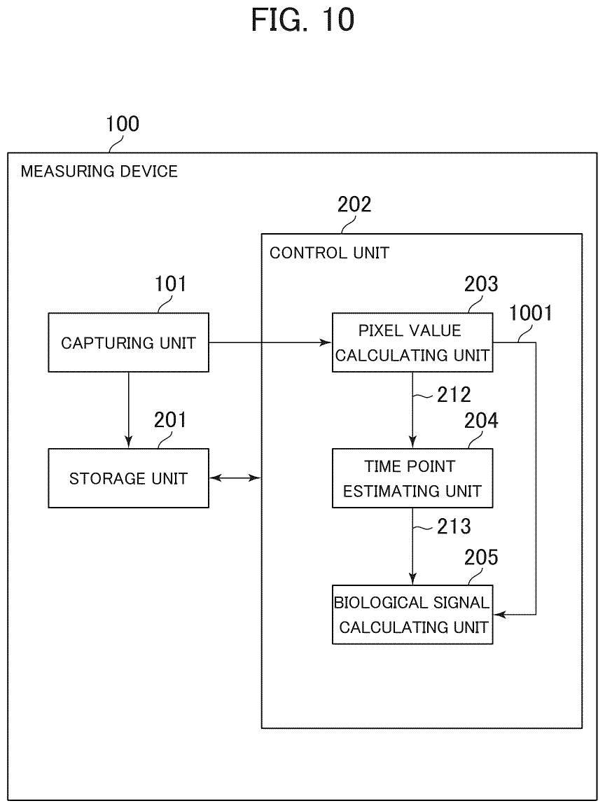

A second embodiment will be described below with reference to FIGS. 10 to 12. Note that, throughout the drawings, like reference signs denote identical or similar constituent features. Such features will not be repeatedly elaborated upon. Configurations and processes having substantially the same functions as those of the first embodiment are denoted by the same reference numerals, and the description thereof will be omitted. Described will be a difference between this embodiment and the first embodiment.

FIG. 10 is a block diagram illustrating an exemplary configuration of the measuring device 100 according to this embodiment. A difference between the measuring device 100 illustrated in FIG. 10 as an example and the measuring device 100 illustrated in FIG. 2 as an example is that, in the measuring device 100 illustrated in FIG. 10 as an example, the pixel value calculating unit 203 further calculates a second representative pixel value 1001; that is, a value of a pixel in a second region of interest.

The pixel value calculating unit 203 according to this embodiment calculates, from each of a plurality of processed images IMG, the first representative pixel value 212 among pixel values of two or more pixels in the first region of interest not including an image of a surface of the biological subject 102. Hence, the pixel value calculating unit 203 calculates each of a plurality of the first representative pixel values 212 from a corresponding one of the plurality of the processed images IMG.

Furthermore, the pixel value calculating unit 203 according to this embodiment calculates, from each of the plurality of processed images IMG, the second representative pixel value 1001 among pixel values of two or more pixels in the second region of interest including an image of a surface of the biological subject 102. Hence, the pixel value calculating unit 203 calculates each of a plurality of the second representative pixel values 1001 from a corresponding one of the plurality of the processed images IMG.

The biological signal calculating unit 205 according to this embodiment calculates a biological signal from the plurality of second representative pixel values 1001.

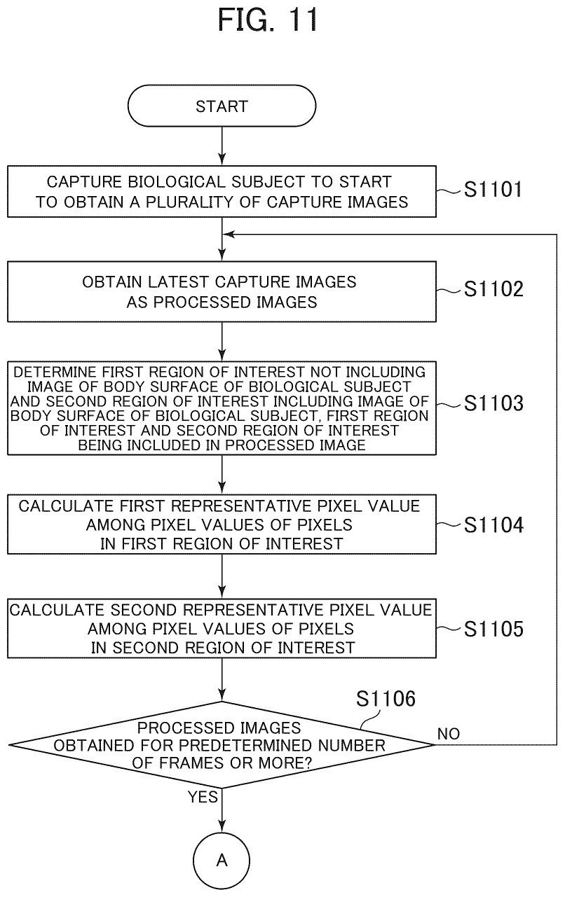

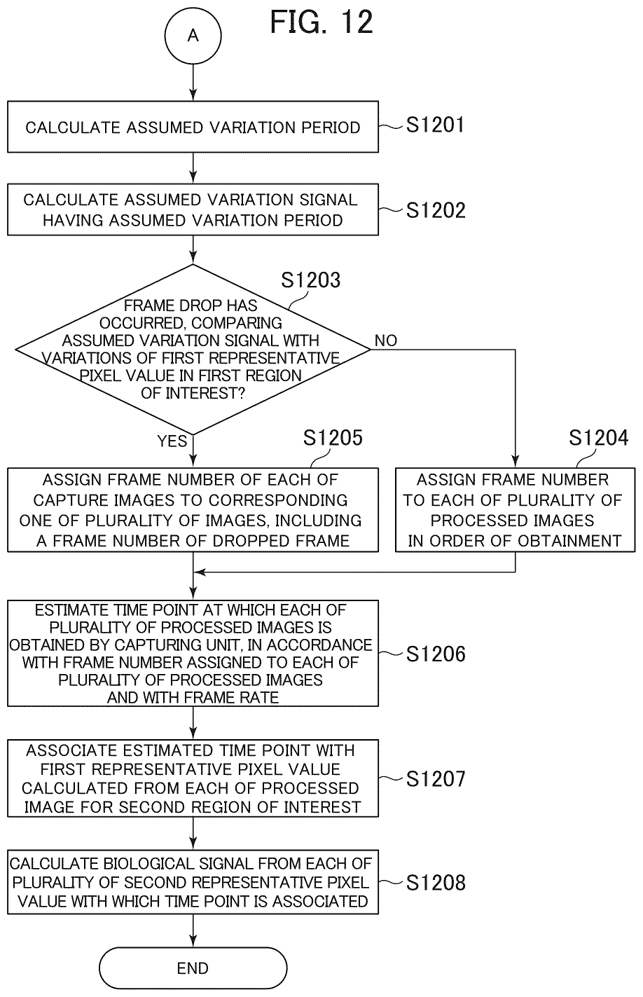

FIGS. 11 and 12 are flowcharts showing an exemplary operation performed on the measuring device 100 according to this embodiment. Processes at Steps S1101 and S1102 illustrated in FIG. 11 as an example are the same as the processes at Steps S301 and S302 illustrated in FIG. 3 as an example, and thus detailed description thereof will be omitted.

At Step S1103, the pixel value calculating unit 203 determines: a first region of interest not including an image of the body surface of the biological subject 102; and a second region of interest including the image of the body surface of the biological subject 102. The first region of interest and the second region of interest are included in a processed image IMG. The first region of interest according to this embodiment indicates an image of a portion in the angle of view for capturing by the capturing unit 101. The portion neither moves nor varies in color. For example, the first region of interest indicates an image of, for example, a wall or a ceiling. The second region of interest is the same as the first region of interest according to the first embodiment, and thus a detailed description thereof will be omitted.

In the image of the body surface of the biological subject 102, the pixel value periodically varies because of aliasing caused by the influence of a lighting fixture that regularly brinks. In addition, the pixel value periodically varies because of variations in the volume of a blood vessel of the biological subject 102. Hence, the image of the portion neither moving nor varying in color shows more clearly variations in periodic pixel value because of aliasing caused by the influence of a lighting fixture that regularly brinks than the image of the body surface of the biological subject

At Step S1104, the pixel value calculating unit 203 calculates the first representative pixel value 212 among pixel values of pixels in the first region of interest determined at Step S1103. A process at Step S1104 is the same as the process at Step S304 illustrated in FIG. 3 as an example, and thus detailed description thereof will be omitted.

At Step S1105, the pixel value calculating unit 203 calculates the second representative pixel value 1001 among pixel values of pixels in the second region of interest determined at Step S1103. For example, the second representative pixel value 1001 is an average value, a median value, or a mode value of the pixel values of the pixels in the second region of interest. For example, if the capturing unit 101 is an image sensor for a camera including a red-green-blue (RGB) filter, the pixel value calculating unit 203 may calculate the second representative pixel value 1001 among the pixel values of the pixels for each of R, G, and B in the second region of interest.

At Step S1106, the pixel value calculating unit 203 determines whether the processed images IMG are obtained for a predetermined number of frames or more. A process at Step S1106 is the same as the process at Step S305 illustrated in FIG. 3 as an example, and thus detailed description thereof will be omitted.

At Step S1106, if the processed images IMG are not obtained for the predetermined number of frames or more, the control unit 202 returns the process back to Step S1102, and continues the process. Whereas, at Step S1106, if the processed images IMG are obtained for the predetermined number of frames or more, the control unit 202 proceeds with the process to Step S1201 exemplified in FIG. 12.

Subsequently described below with reference to FIG. 12 will be the operation of the measuring device 100 according to this embodiment. Processes at Steps S1201 and S1202 illustrated in FIG. 12 as an example are the same as the processes at Steps S401 and S402 illustrated in FIG. 4 as an example, and thus detailed description thereof will be omitted.

At Step S1203, the time point estimating unit 204 compares the assumed variation signal 801 calculated at Step S1202 with the time-series variations of the first representative pixel value 212 to determine whether a frame drop has occurred. That is, the time point estimating unit 204 compares the assumed variation signal 801 and variations of the first representative pixel value 212 calculated from the first region of interest including the image of the portion neither moving nor varying in color, and determines whether the frame drop has occurred. When the first region of interest includes not the image of the body surface of the biological subject 102 but the image of the portion neither moving nor varying in color, the pixel value does not periodically vary because of the variations in the volume of the blood vessel of the biological subject 102. That is why the pixel value in aliasing clearly varies periodically because of the influence of the lighting fixture that regularly blinks, and the time point estimating unit 204 according to this embodiment can determine more accurately whether a frame drop has occurred than the time point estimating unit 204 according to the first embodiment.

At Step S1203, if a frame drop has not occurred, the control unit 202 proceeds with the process to Step S1204. Whereas, at Step S1203, if a frame drop has occurred, the control unit 202 proceeds with the process to Step S1205. Processes at Steps S1204 to S1206 are the same as the processes at Step S404 to S406 illustrated in FIG. 4 as an example, and thus detailed description thereof will be omitted.

The first region of interest according to this embodiment includes, in the angle of view for capturing by the capturing unit 101, not the image of the body surface of the biological subject 102 but the image of the portion neither moving nor varying in color. Thanks to such a feature, because of the time-series variations of the first representative pixel value 212, the time point estimating unit 204 according to this embodiment can estimate the time point 213, at which the processed image IMG is obtained by the capturing unit 101, more accurately than the time point estimating unit 204 according to the first embodiment.

At Step S1207, the time point estimating unit 204 associates the time point 213 with the second representative pixel value 1001 for the second region of interest. Here, the time point 213 is a time point at which the capturing unit 101 obtains the processed image IMG from which the second representative pixel value 1001 is calculated. Then, the control unit 202 proceeds with the process to Step S1208. A process at Step S1208 is the same as the process at Step S408 illustrated in FIG. 4 as an example, and thus detailed description thereof will be omitted.

As can be seen, the measuring device 100 according to this embodiment can associate, with the second representative pixel value 1001, a time point more accurate than a time point that the measuring device 100 according to the first embodiment associates, thereby successfully calculating a biological signal more accurately than the measuring device 100 according to the first embodiment.

### Modification

As a modification of the measuring device 100 according to this embodiment, the first region of interest may include all the pixels of each of the processed images IMG. That is, the pixel value calculating unit 203 according to this embodiment calculates, for each of the plurality of processed images IMG, the first representative pixel value 212 among the pixel values of all the pixels in the processed image IMG. Hence, the measuring device 100 according to this modification calculates the first representative pixel value 212 without executing a process to identify a region that does not include an image of a body surface of the biological subject 102. The measuring device 100 according to this modification can reduce a processing time period required for a process to identify the first region of interest.

Each of the processes executed in the above embodiments shall not be limited to the aspects of the processes exemplified in the embodiments. The functional blocks described above may be implemented as logic circuits (hardware) formed into, for example, integrated circuits, or as software using a CPU. Each of the processes executed in the above embodiments may be executed on a plurality of computers. For example, the processes executed on the control unit 202 may be partially executed on another computer. Alternatively, all the processes may be shared and executed among a plurality of computers.

The present disclosure shall not be limited to the above-described embodiments, and may be replaced with a configuration substantially the same as, a configuration having the same advantageous effects as, or a configuration capable of achieving the same object as, the configurations described in the above-described embodiments. In the present disclosure, the technical aspects disclosed in different embodiments are to be appropriately combined together to implement another embodiment. Such an embodiment shall be included within the technical scope of the present disclosure. Furthermore, the technical aspects disclosed in each embodiment may be combined to achieve a new technical feature.

What is claimed is:

1. A measuring device, comprising:
a capturing unit configured to capture a biological subject to sequentially obtain a plurality of images; and
a time point estimating unit configured to estimate a time point at which each of the plurality of images is obtained in accordance with a periodic variation that appears in a sequence of a plurality of pixel values each obtained from a corresponding one of the plurality of images.

2. The measuring device according to claim 1,
wherein the capturing unit sequentially obtains a plurality of capture images at a frame rate set to cause aliasing due to a blink that a lighting fixture makes regularly, and
the plurality of images are at least partially the plurality of capture images.

3. The measuring device according to claim 2, further comprising
a pixel value calculating unit configured to calculate, from each of the plurality of images, a first representative pixel value among pixel values of two or more pixels in a first region of interest in order to calculate each of a plurality of the first representative pixel values from a corresponding one of the plurality of images,
wherein the plurality of pixel values are the plurality of first representative pixel values.

4. The measuring device according to claim 3,
wherein the time point estimating unit calculates a period at the periodic variation in accordance with the frame rate and a frequency of the blink, and determines that a frame drop has occurred if a signal having the frequency does not match the plurality of first representative pixel values.

5. The measuring device according to claim 2,
wherein the periodic variation includes a frequency component due to the blink.

6. The measuring device according to claim 3,
wherein the first region of interest includes an image of a body surface of the biological subject, and
the measuring device further comprises a biological signal calculating unit configured to calculate a biological signal from the plurality of first representative pixel values.

7. The measuring device according to claim 3,
wherein the first region of interest does not include the image of the body surface of the biological subject,
the pixel value calculating unit calculates, from each of the plurality of images, a second representative pixel value among pixel values of two or more pixels in a second region of interest in order to calculate each of a plurality of the second representative pixel values from a corresponding one of the plurality of images, and
the measuring device further comprises a biological signal calculating unit configured to calculate a biological signal from the plurality of the second representative pixel values.

8. The measuring device according to claim 6,
wherein the biological signal indicates a pulse wave signal.

9. A measuring method, comprising:
a step of capturing a biological subject to sequentially obtain a plurality of images; and
a step of estimating a time point at which each of the plurality of images is obtained in accordance with a periodic variation that appears in a sequence of a plurality of pixel values each obtained from a corresponding one of the plurality of images.

10. The measuring device according to claim 7,
wherein the biological signal indicates a pulse wave signal.
